(19) United States
(12) Patent Application Publication
(10) Pub. No.: US 2006/0200026 A1
(43) Pub. Date: Sep. 7, 2006

(54) Robotic catheter system

(75) Inventors: Daniel T. Wallace, Burlingame, CA (US); Robert G. Younge, Portola Valley, CA (US); Frederic H. Moll, Woodside, CA (US); Federico Barbagli, San Francisco, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 11/331,576

(22) Filed: Jan. 13, 2006

Related U.S. Application Data
(63) Continuation-in-part of application No. 11/176,598, filed on Jul. 6, 2005.
(60) Provisional application No. 60/644,505, filed on Jan. 13, 2005.

(51) Int. Cl. A61B 5/05

(52) U.S. Cl. 600/424

(57) ABSTRACT

A method comprises inserting a flexible instrument in a body; maneuvering the instrument using a robotically controlled system; predicting a location of the instrument in the body using kinematic analysis; generating a graphical reconstruction of the catheter at the predicted location; obtaining an image of the catheter in the body; and comparing the image of the catheter with the graphical reconstruction to determine an error in the predicted location.

### RELATED APPLICATION DATA

This application claims the benefit under 35 U.S.C. §119 of Provisional Application No. 60/644,505, filed Jan. 13, 2005, which is fully incorporated by reference herein. This application is also a continuation-in-part of U.S. patent application Ser. No. 11/176,598, filed Jul. 6, 2005, which is fully incorporated by reference herein.

### FIELD OF THE INVENTION

The field of the invention generally relates to robotic surgical devices and methods.

### BACKGROUND OF THE INVENTION

Telerobotic surgical systems and devices are well suited for use in performing minimally invasive medical procedures, as opposed to conventional techniques wherein the patient's body cavity is open to permit the surgeon's hands access to internal organs. While various systems for conducting medical procedures have been introduced, few have been ideally suited to fit the somewhat extreme and contradictory demands required in many minimally invasive procedures. Thus, there is a need for a highly controllable yet minimally sized system to facilitate imaging, diagnosis, and treatment of tissues which may lie deep within a patient, and which may be preferably accessed only via naturally-occurring pathways such as blood vessels or the gastrointestinal tract.

### SUMMARY OF THE INVENTION

In a first embodiment of the invention, a method includes inserting a flexible instrument in a body. The instrument is maneuvered using a robotically controlled system. The location of the instrument in the body is predicted using kinematic analysis. A graphical reconstruction of the instrument is generated showing the predicted location. An image is obtained of the instrument in the body and the image of the instrument in the body is compared with the graphical reconstruction to determine an error in the predicted location.

In another aspect of the invention, a method of graphically displaying the position of a surgical instrument coupled to a robotic system includes acquiring substantially real-time images of the surgical instrument and determining a predicted position of the surgical instrument based on one or more commanded inputs to the robotic system. The substantially real-time images are displayed on a display. The substantially real-time images are overlaid with a graphical rendering of the predicted position of the surgical instrument on the display.

In another aspect of the invention, a system for graphically displaying the position of a surgical instrument coupled to a robotic system includes a fluoroscopic imaging system, an image acquisition system, a control system for controlling the position of the surgical instrument, and a display for simultaneously displaying images of the surgical instrument obtained from the fluoroscopic imaging system and a graphical rendering of the predicted position of the surgical instrument based on one or more inputs to the control system.

### BRIEF DESCRIPTION OF THE DRAWINGS

The present invention is illustrated by way of example and is not limited in the figures of the accompanying drawings, in which like references indicate similar elements. Features shown in the drawings are not intended to be drawn to scale, nor are they intended to be shown in precise positional relationship.

FIG. 1 illustrates a robotic surgical system in accordance with an embodiment of the invention.

FIG. 2 schematically illustrates a control system according to an embodiment of the invention.

FIG. 3A illustrates a robotic catheter system according to an embodiment of the invention.

FIG. 3B illustrates a robotic catheter system according to another embodiment of the invention.

FIG. 4 illustrates a digitized “dashboard” or “windshield” display to enhance instinctive drivability of the pertinent instrumentation within the pertinent tissue structures.

FIG. 5 illustrates a system for overlaying real-time fluoroscopy images with digitally-generated “cartoon” representations of the predicted locations of various structures or images.

FIG. 6 illustrates an exemplary display illustrating a cartoon rendering of a guide catheter's predicted or commanded instrument position overlaid in front of the fluoroscopy plane.

FIG. 7 illustrates another exemplary display illustrating a cartoon rendering of a guide catheter's predicted or commanded instrument position overlaid in front of the fluoroscopy plane.

FIG. 8 is a schematic representation of a system for displaying overlaid images according to one embodiment of the invention.

FIG. 9 illustrates forward kinematics and inverse kinematics in accordance with an embodiment of the invention.

FIG. 10 illustrates task coordinates, joint coordinates, and actuation coordinates in accordance with an embodiment of the invention.

FIG. 11 illustrates variables associated with a geometry of a catheter in accordance with one embodiment of the invention.

### DETAILED DESCRIPTION

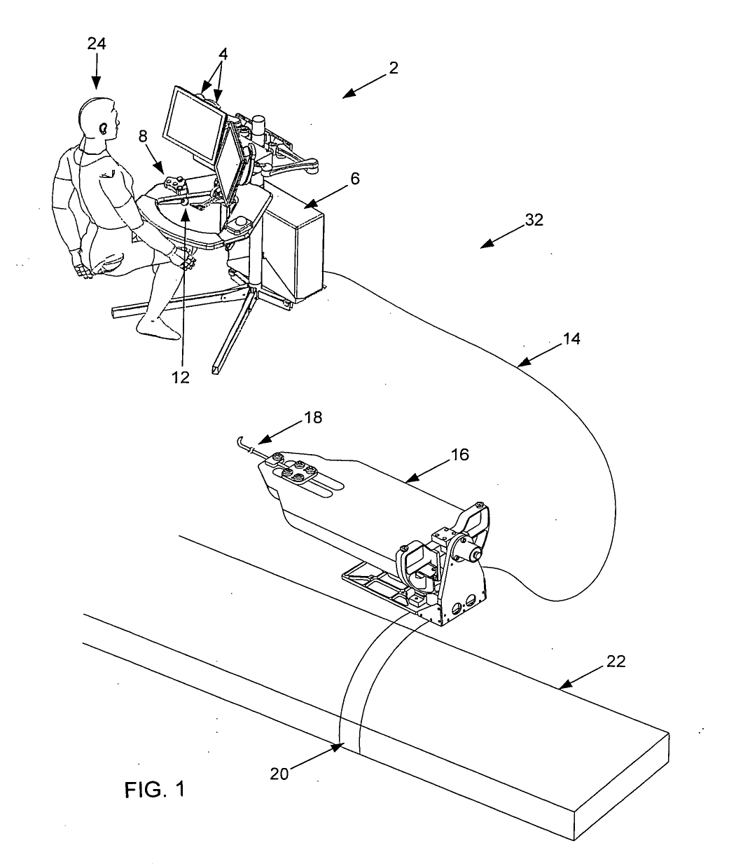

Referring to FIG. 1, one embodiment of a robotic surgical system (32) is depicted having an operator control station (2) located remotely from an operating table (22), to which a instrument driver (16) and instrument (18) are coupled by a instrument driver mounting brace (20). A wired connection (14) transfers signals between the operator control station (2) and instrument driver (16). The instrument driver mounting brace (20) of the depicted embodiment is a relatively simple arcuate-shaped structural member configured to position the instrument driver (16) above a patient (not shown) lying on the table below (22). Various embodiments of the surgical system 32 are disclosed and described in detail in the above-incorporated U.S. application Ser. No. 11/176,598.

As is also described in application Ser. No. 11/176,598, visualization software provides an operator at an operator control station (2), such as that depicted in FIG. 1, with a digitized “dashboard” or “windshield” display to enhance instinctive drivability of the pertinent instrumentation within the pertinent tissue structures.

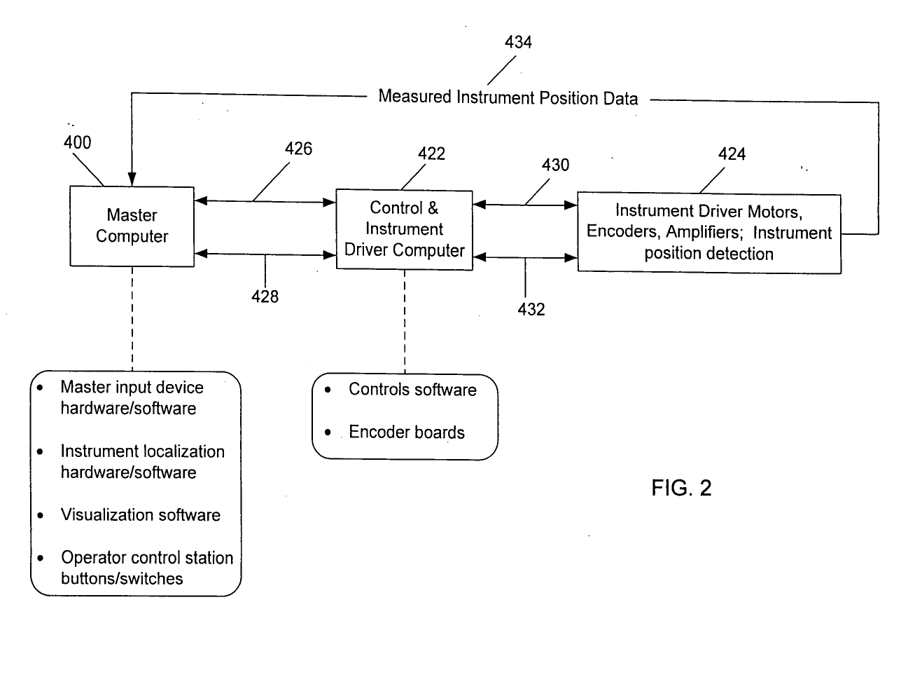

Referring to FIG. 2, an overview of an embodiment of a controls system flow is depicted. The depicted embodiment comprises a master computer (400) running master input device software, visualization software, instrument localization software, and software to interface with operator control station buttons and/or switches. In one embodiment, the master input device software is a proprietary module packaged with an off-the-shelf master input device system, such as the Phantom™ from Sensible Devices Corporation, which is configured to communicate with the Phantom™ hardware at a relatively high frequency as prescribed by the manufacturer. The master input device (12) may also have haptics capability to facilitate feedback to the operator, and the software modules pertinent to such functionality may also be operated on the master computer (100). Preferred embodiments of haptics feedback to the operator are discussed in further detail below.

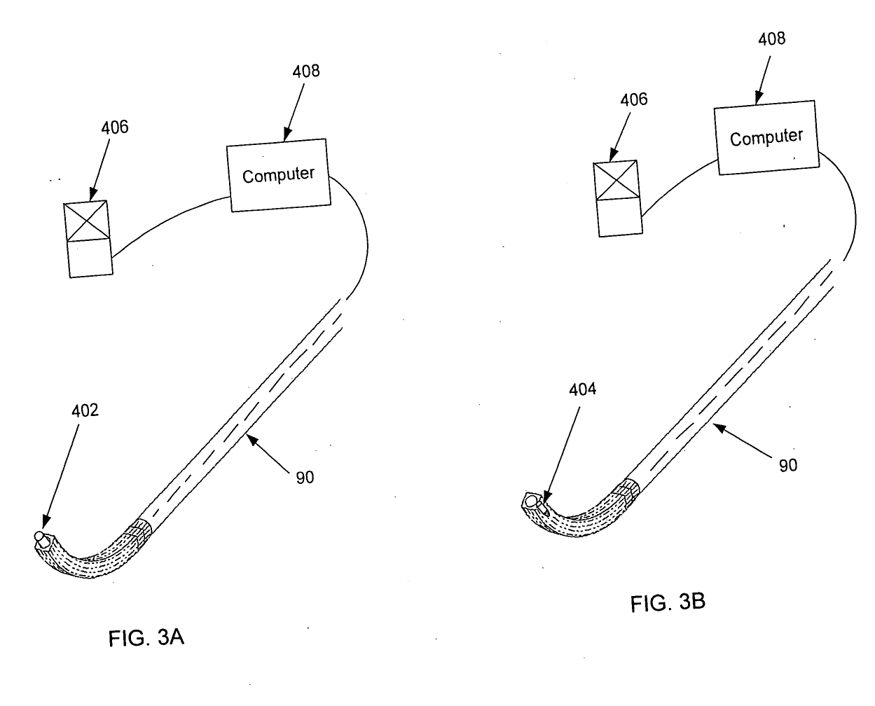

The term “localization” is used in the art in reference to systems for monitoring the position of objects, such as medical instruments, in space. In one embodiment, the instrument localization software is a proprietary module packaged with an off-the-shelf or custom instrument position tracking system, such as those available from Ascension Technology Corporation, Biosense Webster Corporation, and others. Referring to FIGS. 3A and 3B, conventional localization sensing systems such as these may be utilized with the subject robotic catheter system in various embodiments. As shown in FIG. 3A, one preferred localization system comprises an electromagnetic field transmitter (406) and an electromagnetic field receiver (402) positioned within the central lumen of a guide catheter (90). The transmitter (406) and receiver (402) are interfaced with a computer operating software configured to detect the position of the detector relative to the coordinate system of the transmitter (406) in real or near-real time with high degrees of accuracy.

Referring to FIG. 3B, a similar embodiment is depicted with a receiver (404) embedded within the guide catheter (90) construction. Preferred receiver structures may comprise three or more sets of very small coils spatially configured to sense orthogonal aspects of magnetic fields emitted by a transmitter. Such coils may be embedded in a custom configuration within or around the walls of a preferred catheter construct. For example, in one embodiment, two orthogonal coils are embedded within a thin polymeric layer at two slightly flattened surfaces of a catheter (90) body approximately 90 degrees orthogonal to each other about the longitudinal axis of the catheter (90) body, and a third coil is embedded in a slight polymer-encapsulated protrusion from the outside of the catheter (90) body, perpendicular to the other two coils. Due to the very small size of the pertinent coils, the protrusion of the third coil may be minimized. Electronic leads for such coils may also be embedded in the catheter wall, down the length of the catheter body to a position, preferably adjacent an instrument driver, where they may be routed away from the instrument to a computer running localization software and interfaced with a pertinent transmitter.

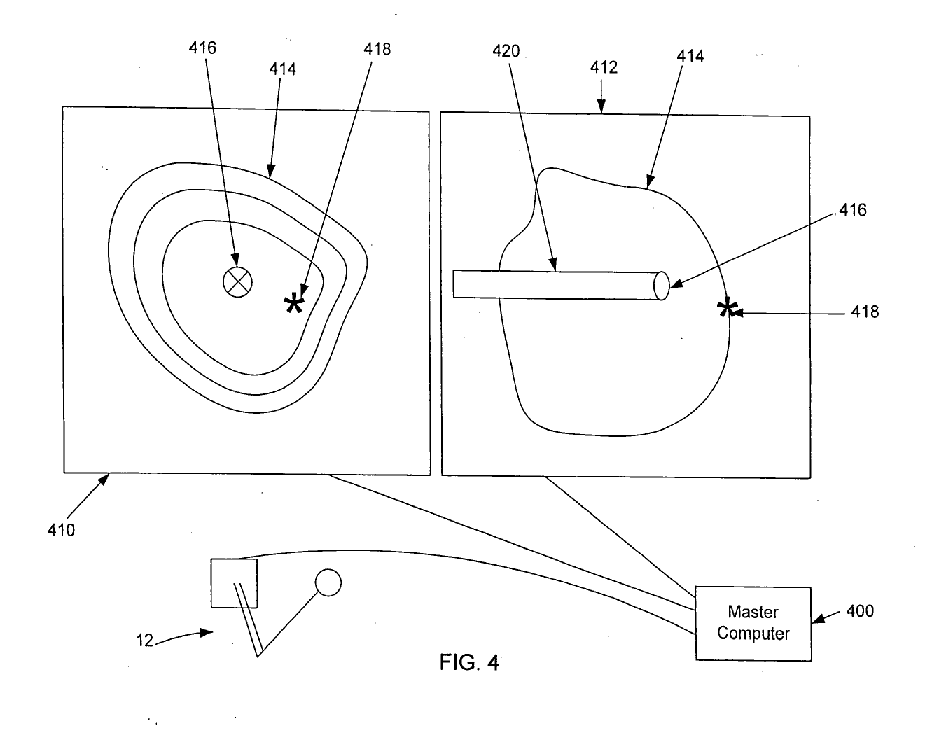

Referring back to FIG. 2, in one embodiment, visualization software runs on the master computer (400) to facilitate real-time driving and navigation of one or more steerable instruments. In one embodiment, visualization software provides an operator at an operator control station (2), such as that depicted in FIG. 1, with a digitized “dashboard” or “windshield” display to enhance instinctive drivability of the pertinent instrumentation within the pertinent tissue structures. Referring to FIG. 4, a simple illustration is useful to explain one embodiment of a preferred relationship between visualization and navigation with a master input device (12). In the depicted embodiment, two display views (410, 412) are shown. One preferably represents a primary (410) navigation view, and one may represent a secondary (412) navigation view. To facilitate instinctive operation of the system, it is preferable to have the master input device coordinate system at least approximately synchronized with the coordinate system of at least one of the two views. Further, it is preferable to provide the operator with one or more secondary views which may be helpful in navigating through challenging tissue structure pathways and geometries.

Using the operation of an automobile as an example, if the master input device is a steering wheel and the operator desires to drive a car in a forward direction using one or more views, his first priority is likely to have a view straight out the windshield, as opposed to a view out the back window, out one of the side windows, or from a car in front of the car that he is operating. In such an example, the operator might prefer to have the forward windshield view as his primary display view—so a right turn on the steering wheel take him right as he observes his primary display, a left turn on the steering wheel manifests itself in his primary display as turn to the left, etc—instinctive driving or navigation. If the operator of the automobile is trying to park his car adjacent another car parked directly in front of him, it might be preferable to also have a view from a camera positioned, for example, upon the sidewalk aimed perpendicularly through the space between the two cars (one driven by the operator and one parked in front of the driven car)—so the operator can see the gap closing between his car and the car in front of him as he parks. While the driver might not prefer to have to completely operate his vehicle with the sidewalk perpendicular camera view as his sole visualization for navigation purposes, this view is helpful as a secondary view.

Referring back to FIG. 4, if an operator is attempting to navigate a steerable catheter to, for example, touch the catheter's distal tip upon a particular tissue location, a useful primary navigation view (410) comprises a three dimensional digital model of the pertinent tissue structures (414) through which the operator is navigating the catheter with the master input device (12), and a representation of the catheter distal tip location (416) as viewed along the longitudinal axis of the catheter near the distal tip. The depicted embodiment also illustrates a representation of a targeted tissue structure location (418) which may be desired in addition to the tissue digital model (414) information. A useful secondary view (412), displayed upon a different monitor, in a different window upon the same monitor, or within the same user interface window, for example, comprises an orthogonal view depicting the catheter tip representation (416), and also perhaps a catheter body representation (420), to facilitate the operator's driving of the catheter tip toward the desired targeted tissue location (418).

In one embodiment, subsequent to development and display of a digital model of pertinent tissue structures, an operator may select one primary and at least one secondary view to facilitate navigation of the instrumentation. In one embodiment, by selecting which view is a primary view, the user automatically toggles master input device (12) coordinate system to synchronize with the selected primary view. Referring again to FIG. 4, in such an embodiment with the leftmost depicted view (410) selected as the primary view, to navigate toward the targeted tissue site (418), the operator should manipulate the master input device (12) forward, to the right, and down. The right view will provide valued navigation information, but will not be as instinctive from a “driving” perspective.

To illustrate this non-instinctiveness, if in the depicted example the operator wishes to insert the catheter tip toward the targeted tissue site (418) watching only the rightmost view (412) without the master input device (12) coordinate system synchronized with such view, the operator would have to remember that pushing straight ahead on the master input device will make the distal tip representation (416) move to the right on the rightmost display (412). Should the operator decide to toggle the system to use the rightmost view (412) as the primary navigation view, the coordinate system of the master input device (12) is then synchronized with that of the rightmost view (412), enabling the operator to move the catheter tip (416) closer to the desired targeted tissue location (418) by manipulating the master input device (12) down and to the right.

It may be useful to present the operator with one or more views of various graphical objects in an overlaid format, to facilitate the user's comprehension of relative positioning of the various structures. For example, it maybe useful to overlay a real-time fluoroscopy image with digitally-generated “cartoon” representations of the predicted locations of various structures or images. Indeed, in one embodiment, a real-time or updated-as-acquired fluoroscopy image including a fluoroscopic representation of the location of an instrument may be overlaid with a real-time representation of where the computerized system expects the instrument to be relative to the surrounding anatomy.

In a related variation, updated images from other associated modalities, such as intracardiac echo ultrasound (“ICE”), may also be overlaid onto the display with the fluoro and instrument “cartoon” image, to provide the operator with an information-rich rendering on one display.

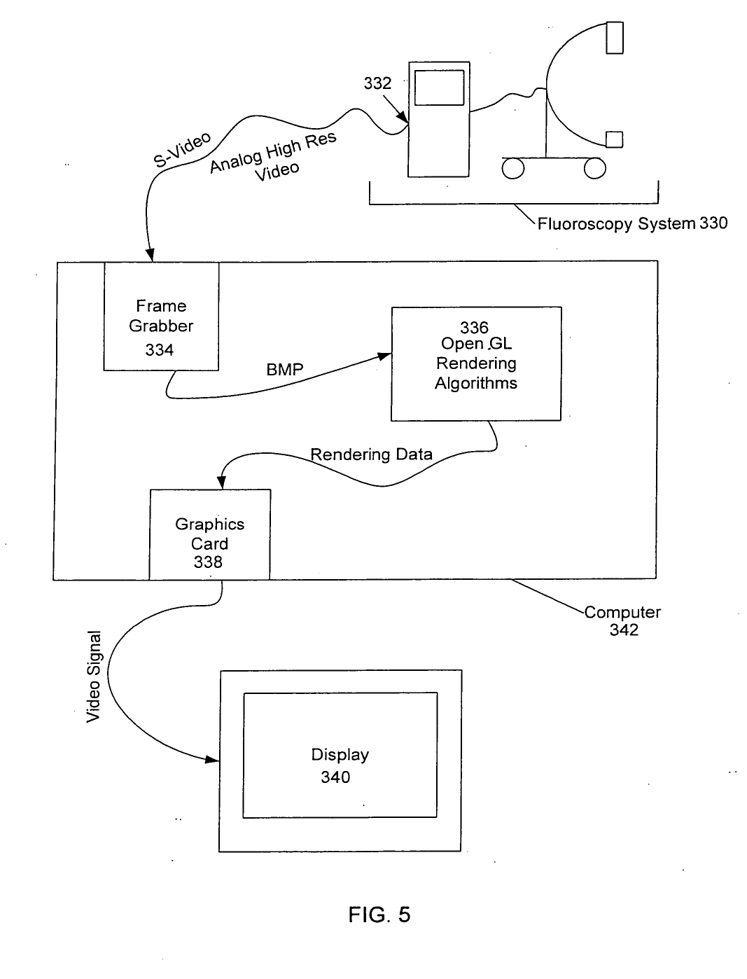

Referring to FIG. 5, a systemic view configured to produce such an overlaid image is depicted. As shown in FIG. 5, a conventional fluoroscopy system (330) outputs an electronic image in formats such as those known as “S-video” or “analog high-resolution video”. In image output interface (332) of a fluoroscopy system (330) may be connected to an input interface of a computer (342) based image acquisition device, such as those known as “frame grabber” (334) image acquisition cards, to facilitate intake of the video signal from the fluoroscopy system (330) into the frame grabber (334), which may be configured to produce bitmap (“BMP”) digital image data, generally comprising a series of Cartesian pixel coordinates and associated grayscale or color values which together may be depicted as an image. The bitmap data may then be processed utilizing computer graphics rendering algorithms, such as those available in conventional “OpenGL” graphics libraries (336).

In summary, conventional OpenGL functionality enables a programmer or operator to define object positions, textures, sizes, lights, and cameras to produce three-dimensional renderings on a two-dimensional display. The process of building a scene, describing objects, lights, and camera position, and using OpenGL functionality to turn such a configuration into a two-dimensional image for display is known in computer graphics as “rendering”. The description of objects may be handled by forming a mesh of triangles, which conventional graphics cards are configured to interpret and output displayable two-dimensional images for a conventional display or computer monitor, as would be apparent to one skilled in the art. Thus the OpenGL software (336) may be configured to send rendering data to the graphics card (338) in the system depicted in FIG. 5, which may then be output to a conventional display (340).

In one embodiment, a triangular mesh generated with OpenGL software to form a cartoon-like rendering of an elongate instrument moving in space according to movements from, for example, a master following mode operational state, may be directed to a computer graphics card, along with frame grabber and OpenGL processed fluoroscopic video data. Thus a moving cartoon-like image of an elongate instrument would be displayable. To project updated fluoroscopic image data onto a flat-appearing surface in the same display, a plane object, conventionally rendered by defining two triangles, may be created, and the updated fluoroscopic image data may be texture mapped onto the plane. Thus the cartoon-like image of the elongate instrument may be overlaid with the plane object upon which the updated fluoroscopic image data is texture mapped. Camera and light source positioning may be pre-selected, or selectable by the operator through the mouse or other input device, for example, to enable the operator to select desired image perspectives for his two-dimensional computer display.

The perspectives, which may be defined as origin position and vector position of the camera, may be selected to match with standard views coming from a fluoroscopy system, such as anterior/posterior and lateral views of a patient lying on an operating table. When the elongate instrument is visible in the fluoroscopy images, the fluoroscopy plane object and cartoon instrument object may be registered with each other by ensuring that the instrument depicted in the fluoroscopy plane lines up with the cartoon version of the instrument. In one embodiment, several perspectives are viewed while the cartoon object is moved using an input device such as a mouse, until the cartoon instrument object is registered with the fluoroscopic plane image of the instrument. Since both the position of the cartoon object and fluoroscopic image object may be updated in real time, an operator, or the system automatically through image processing of the overlaid image, may interpret significant depicted mismatch between the position of the instrument cartoon and the instrument fluoroscopic image as contact with a structure that is inhibiting the normal predicted motion of the instrument, error or malfunction in the instrument, or error or malfunction in the predictive controls software underlying the depicted position of the instrument cartoon.

Referring back to FIG. 5, other video signals (not shown) may be directed to the image grabber (334), besides that of a fluoroscopy system (330), simultaneously. For example, images from an intracardiac echo ultrasound (“ICE”) system, intravascular ultrasound (“IVUS”), or other system may be overlaid onto the same displayed image simultaneously. Further, additional objects besides a plane for texture mapping fluoroscopy or a elongate instrument cartoon object may be processed using OpenGL or other rendering software to add additional objects to the final display.

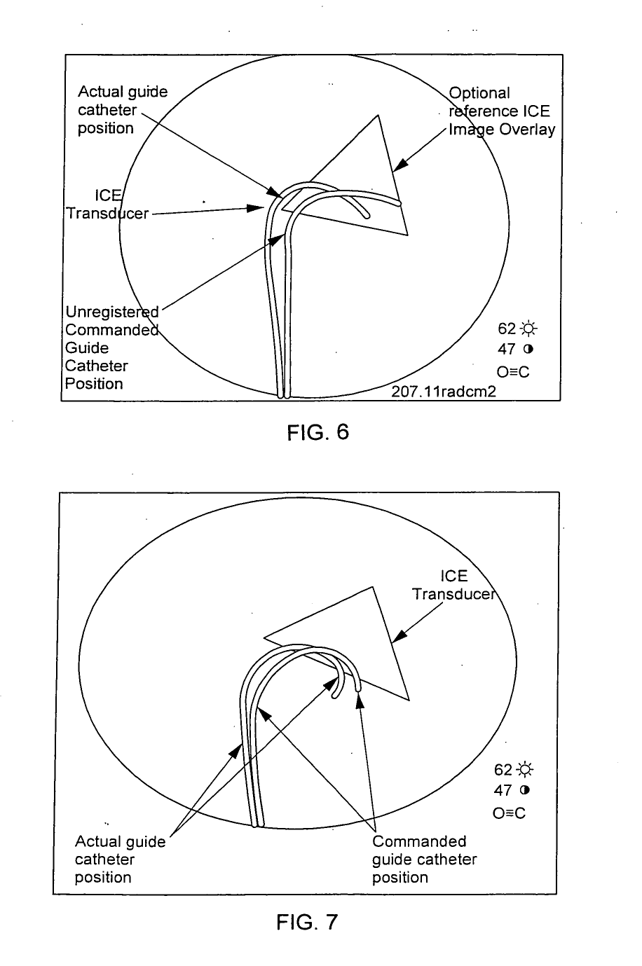

Referring to FIGS. 6-8, one embodiment is illustrated wherein the elongate instrument is a robotic guide catheter, and fluoroscopy and ICE are utilized to visualize the cardiac and other surrounding tissues, and instrument objects. Referring to FIG. 6, a fluoroscopy image has been texture mapped upon a plane configured to occupy nearly the entire display area in the background. Visible in the fluoroscopy image as a dark elongate shadow is the actual position, from fluoroscopy, of the guide catheter instrument relative to the surrounding tissues overlaid in front of the fluoroscopy plane is a cartoon rendering (white in color in FIGS. 6 and 7) of the predicted, or “commanded”, guide catheter instrument position. Further overlaid in front of the fluoroscopy plane is a small cartoon object representing the position of the ICE transducer, as well as another plane object adjacent the ICE transducer cartoon object onto which the ICE image data is texture mapped by a technique similar to that with which the fluoroscopic images are texture mapped upon the background plane object. Further, mouse objects, software menu objects, and many other objects may be overlaid. FIG. 7 shows a similar view with the instrument in a different position. For illustrative purposes, FIGS. 6 and 7 depict misalignment of the instrument position from the fluoroscopy object, as compared with the instrument position from the cartoon object. As described above, the various objects may be registered to each other by manually aligning cartoon objects with captured image objects in multiple views until the various objects are aligned as desired. Image processing of markers and shapes of various objects may be utilized to automate portions of such a registration process.

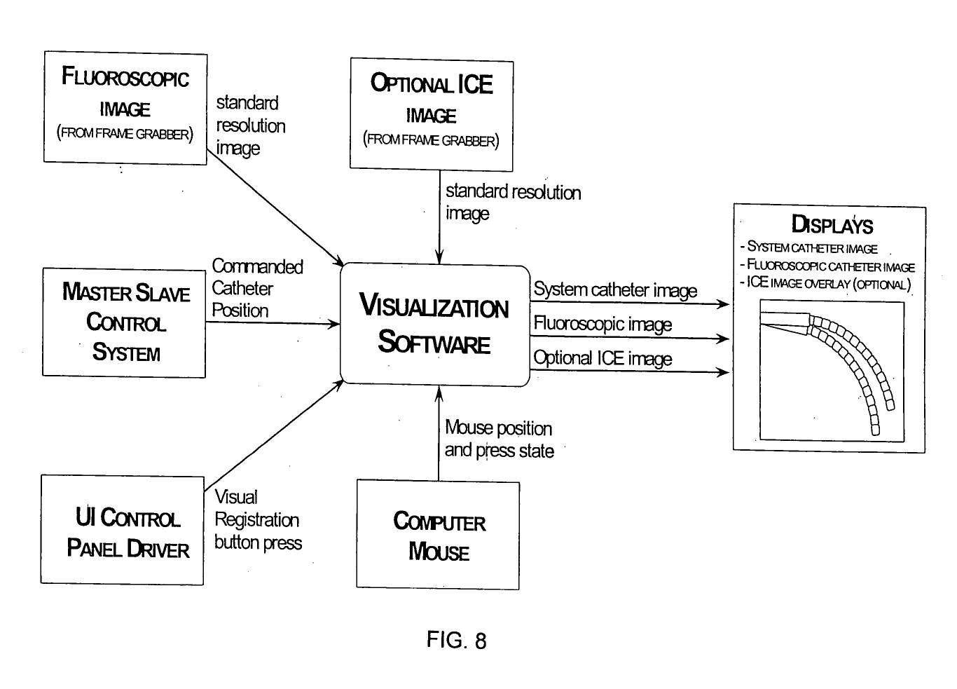

Referring to FIG. 8, a schematic is depicted to illustrate how various objects, originating from actual medical images processed by frame grabber, originating from commanded instrument position control outputs, or originating from computer operating system visual objects, such as mouse, menu, or control panel objects, may be overlaid into the same display.

In another embodiment, a preacquired image of pertinent tissue, such as a three-dimensional image of a heart, may be overlaid and registered to updated images from real-time imaging modalities as well. For example, in one embodiment, a beating heart may be preoperatively imaged using gated computed tomography (“CT”). The result of CT imaging may be a stack of CT data slices. Utilizing either manual or automated thresholding techniques, along with interpolation, smoothing, and or other conventional image processing techniques available in software packages such as that sold under the trade name Amira™, a triangular mesh may be constructed to represent a three-dimensional cartoon-like object of the heart, saved, for example, as an object (“.obj”) file, and added to the rendering as a heart object. The heart object may then be registered as discussed above to other depicted images, such as fluoroscopy images, utilizing known tissue landmarks in multiple views, and contrast agent techniques to particularly see show certain tissue landmarks, such as the outline of an aorta, ventricle, or left atrium. The cartoon heart object may be moved around, by mouse, for example, until it is appropriately registered in various views, such as anterior/posterior and lateral, with the other overlaid objects.

In one embodiment, interpreted master following interprets commands that would normally lead to dragging along the tissue structure surface as commands to execute a succession of smaller hops to and from the tissue structure surface, while logging each contact as a new point to add to the tissue structure surface model. Hops are preferably executed by backing the instrument out the same trajectory it came into contact with the tissue structure, then moving normally along the wall per the tissue structure model, and reapproaching with a similar trajectory. In addition to saving to memory each new XYZ surface point, in one embodiment the system saves the trajectory of the instrument with which the contact was made by saving the localization orientation data and control element tension commands to allow the operator to re-execute the same trajectory at a later time if so desired. By saving the trajectories and new points of contact confirmation, a more detailed contour map is formed from the tissue structure model, which may be utilized in the procedure and continually enhanced. The length of each hop may be configured, as well as the length of non-contact distance in between each hop contact. Saved trajectories and points of contact confirmation may be utilized to later returns of the instrument to such locations.

For example, in one embodiment, an operator may navigate the instrument around within a cavity, such as a heart chamber, and select certain desirable points to which he may later want to return the instrument. The selected desirable points may be visually marked in the graphical user interface presented to the operator by small colorful marker dots, for example. Should the operator later wish to return the instrument to such points, he may select all of the marked desirable points, or a subset thereof, with a mouse, master input device, keyboard or menu command, or other graphical user interface control device, and execute a command to have the instrument move to the selected locations and perhaps stop in contact at each selected location before moving to the next. Such a movement schema may be utilized for applying energy and ablating tissue at the contact points, as in a cardiac ablation procedure. Movement of the instrument upon the executed command may be driven by relatively simple logic, such as logic which causes the distal portion of the instrument to move in a straight-line pathway to the desired selected contact location, or may be more complex, wherein a previously-utilized instrument trajectory may be followed, or wherein the instrument may be navigated to purposely avoid tissue contact until contact is established with the desired contact location, using geometrically associated anatomic data, for example.

The kinematic relationships for many catheter instrument embodiments may be modeled by applying conventional mechanics relationships. In summary, a control-element-steered catheter instrument is controlled through a set of actuated inputs. In a four-control-element catheter instrument, for example, there are two degrees of motion actuation, pitch and yaw, which both have + and − directions. Other motorized tension relationships may drive other instruments, active tensioning, or insertion or roll of the catheter instrument. The relationship between actuated inputs and the catheter's end point position as a function of the actuated inputs is referred to as the “kinematics” of the catheter.

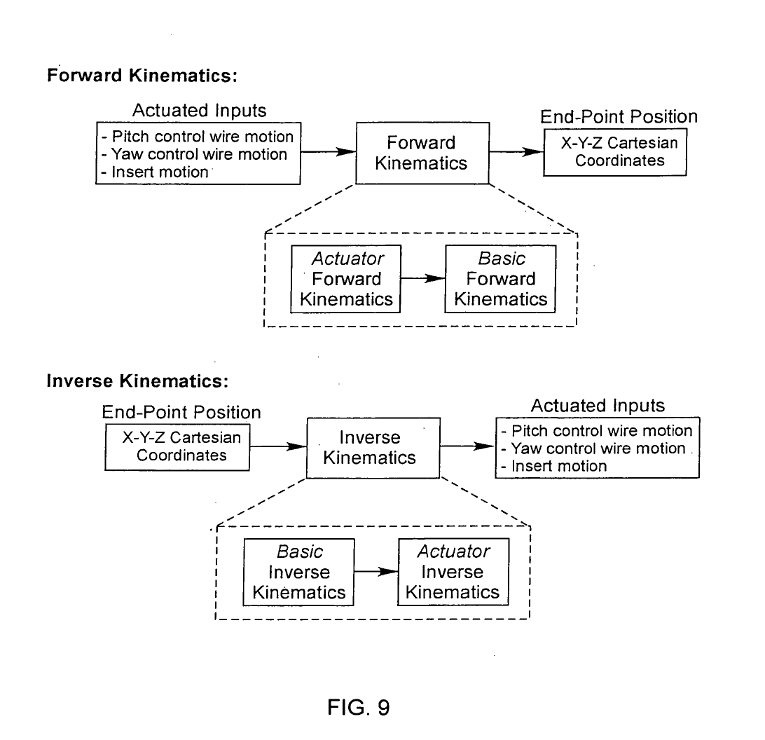

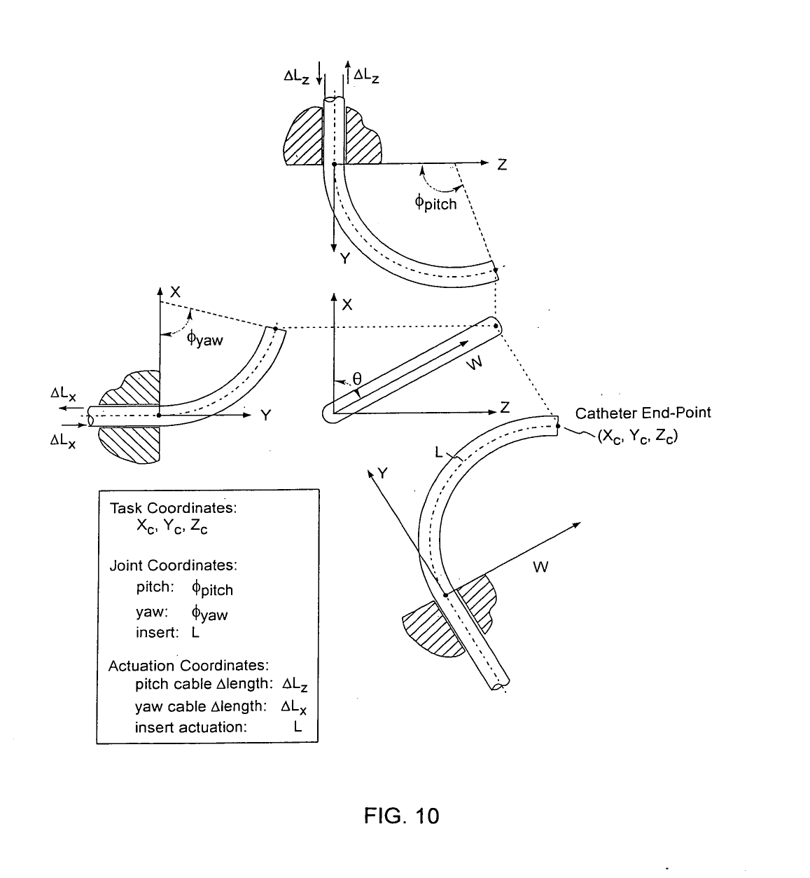

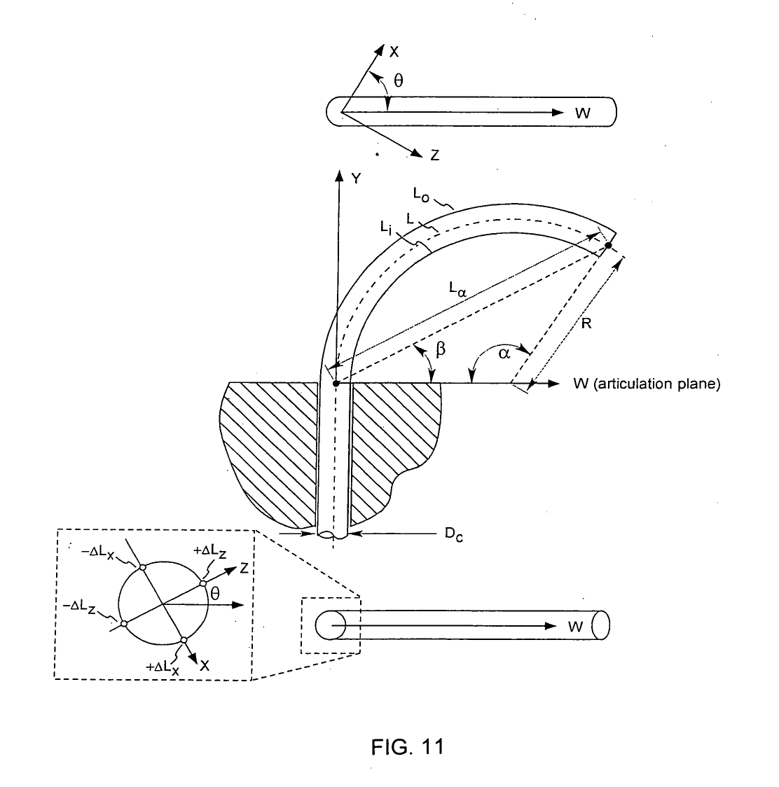

Referring to FIG. 9, the “forward kinematics” expresses the catheter's end-point position as a function of the actuated inputs while the “inverse kinematics” expresses the actuated inputs as a function of the desired end-point position. Accurate mathematical models of the forward and inverse kinematics are essential for the control of a robotically controlled catheter system. For clarity, the kinematics equations are further refined to separate out common elements, as shown in FIG. 9. The basic kinematics describes the relationship between the task coordinates and the joint coordinates. In such case, the task coordinates refer to the position of the catheter end-point while the joint coordinates refer to the bending (pitch and yaw, for example) and length of the active catheter. The actuator kinematics describes the relationship between the actuation coordinates and the joint coordinates. The task, joint, and bending actuation coordinates for the robotic catheter are illustrated in FIG. 10. By describing the kinematics in this way we can separate out the kinematics associated with the catheter structure, namely the basic kinematics, from those associated with the actuation methodology.

The development of the catheter's kinematics model is derived using a few essential assumptions. Included are assumptions that the catheter structure is approximated as a simple beam in bending from a mechanics perspective, and that control elements, such as thin tension wires, remain at a fixed distance from the neutral axis and thus impart a uniform moment along the length of the catheter.

In addition to the above assumptions, the geometry and variables shown in FIG. 11 are used in the derivation of the forward and inverse kinematics. The basic forward kinematics, relating the catheter task coordinates (X_{c}, Y_{c}, Z_{c}) to the joint coordinates (φ_{pitch}, φ_{pitch}, L), is given as follows:

$${X}_{c} = w cos ( θ )$$

$${Y}_{c} = R sin ( α )$$

$${Z}_{c} = w sin ( θ )$$

$$where$$

$$w = R ( 1 - cos ( α ) )$$

$$α = {[ {( {ϕ}_{pitch} )}^{2} + {( {ϕ}_{yaw} )}^{2} ]}^{1 / 2} ( total bending )$$

$$R = \frac{L}{α} ( bend radius )$$

$$θ = a tan 2 ( {ϕ}_{pitch} , {ϕ}_{yaw} ) ( roll angle )$$

The actuator forward kinematics, relating the joint coordinates (φ_{pitch}, φ_{pitch}, L) to the actuator coordinates (ΔLₓ, ΔL_{z}, L) is given as follows:

$${ϕ}_{pitch} = \frac{2 Δ {L}_{z}}{{D}_{c}}$$

$${ϕ}_{yaw} = \frac{2 Δ {L}_{z}}{{D}_{c}}$$

As illustrated in FIG. 9, the catheter's end-point position can be predicted given the joint or actuation coordinates by using the forward kinematics equations described above.

Calculation of the catheter's actuated inputs as a function of end-point position, referred to as the inverse kinematics, can be performed numerically, using a nonlinear equation solver such as Newton-Raphson. A more desirable approach, and the one used in this illustrative embodiment, is to develop a closed-form solution which can be used to calculate the required actuated inputs directly from the desired end-point positions.

As with the forward kinematics, we separate the inverse kinematics into the basic inverse kinematics, which relates joint coordinates to the task coordinates, and the actuation inverse kinematics, which relates the actuation coordinates to the joint coordinates. The basic inverse kinematics, relating the joint coordinates (φ_{pitch}, φ_{pitch}, L), to the catheter task coordinates

$${ϕ}_{pitch} = α sin ( θ )$$

$${ϕ}_{yaw} = α cos ( θ )$$

$$L = R α \\ \to where \to \to \begin{matrix}θ = a tan 2 ( {Z}_{c} , {X}_{c} ) \\ R = \frac{l sin β}{sin 2 β} \\ α = π - 2 β\end{matrix} \to \begin{matrix}β = a tan 2 ( {Y}_{c} , {W}_{c} ) \\ {W}_{c} = {( {X}_{c}^{2} + {Z}_{c}^{2} )}^{1 / 2} \\ l = {( {W}_{c}^{2} + {Y}_{c}^{2} )}^{1 / 2}\end{matrix}$$

The actuator inverse kinematics, relating the actuator coordinates (ΔL, ΔL, L) to the joint coordinates (φ_{pitch}, φ_{pitch}, L) is given as follows:

$$Δ {L}_{x} = \frac{{D}_{c} {ϕ}_{yaw}}{2}$$

$$Δ {L}_{z} = \frac{{D}_{c} {ϕ}_{pitch}}{2}$$

1. A method, comprising:
inserting a flexible instrument in a body;
maneuvering the instrument using a robotically controlled system;
predicting a location of the instrument in the body using kinematic analysis;
generating a graphical reconstruction of the instrument at the predicted location;
obtaining an image of the instrument in the body; and
comparing the image of the instrument with the graphical reconstruction to determine an error in the predicted location.

2. The method of claim 1, further comprising displaying the generated graphical reconstruction and image of the instrument on a display.

3. The method of claim 2, further comprising displaying an intracardiac echo ultrasound (ICE) on the display.

4. The method of claim 2, wherein multiple perspective views of the generated graphical reconstruction and image of the instrument are displayed on the display.

5. The method of claim 2, further comprising overlaying a pre-acquired image of tissue on the display.

6. The method of claim 1, wherein the image of the instrument is a fluoroscopic image.

7. The method of claim 6, wherein the fluoroscopic image is texture mapped upon an image plane.

8. The method of claim 1, wherein the instrument comprises a catheter.

9. A method of graphically displaying the position of a surgical instrument coupled to a robotic system comprising:
acquiring substantially real-time images of the surgical instrument;
determining a predicted position of the surgical instrument based on one or more commanded inputs to the robotic system;
displaying the substantially real-time images on a display; and
overlaying the substantially real-time images with a graphical rendering of the predicted position of the surgical instrument on the display.

10. The method of claim 9, further comprising displaying an intracardiac echo ultrasound (ICE) on the display.

11. The method of claim 9, wherein multiple perspective views of the generated graphical reconstruction and image of the instrument are displayed on the display.

12. The method of claim 9, further comprising overlaying a pre-acquired image of tissue on the display.

13. The method of claim 12, wherein the pre-acquired image comprises a three-dimensional image of a heart.

14. The method of claim 9, wherein the substantially real-time images and the graphical rendering of the surgical instrument are registered with one another.

15. The method of claim 9, further comprising alerting the user to an error or malfunction based at least in part on the degree of mismatch between the substantially real-time images and the graphical rendering of the surgical instrument.

16. A system for graphically displaying the position of a surgical instrument coupled to a robotic system comprising:
a fluoroscopic imaging system;
an image acquisition system;
a control system for controlling the position of the surgical instrument; and
a display for simultaneously displaying images of the surgical instrument obtained from the fluoroscopic imaging system and a graphical rendering of the predicted position of the surgical instrument based on one or more inputs to the control system.

17. The system according to claim 16, wherein the surgical instrument comprises a catheter.

18. The system according to claim 16, wherein the display also simultaneously displays an intracardiac echo ultrasound (ICE) image.

19. The system according to claim 16, further comprising an error detector that automatically detects an error or malfunction based at least in part on the degree of mismatch between the fluoroscopic images and the graphical rendering of the surgical instrument.

20. The system according to claim 16, wherein the display also simultaneously displays a pre-acquired image of tissue.
